(19) United States
(12) Patent Application Publication
(10) Pub. No.: US 2004/0267045 A1
(43) Pub. Date: Dec. 30, 2004

(54) Processes for producing (meth)acrylic acid compound

(75) Inventors: Shuhei Yada, Mie (JP); Kenji Takasaki, Mie (JP); Yasushi Ogawa, Mie (JP); Yoshiro Suzuki, Mie (JP)

Correspondence Address: OBLON, SPIVAK, MCCLELLAND, MAIER & NEUSTADT, P.C., 1940 DUKE STREET, ALEXANDRIA, VA, 22314, US

(73) Assignee: MITSUBISHI CHEMICAL CORPORATION, Tokyo (JP)

(21) Appl. No.: 10/854,704

(22) Filed: May 27, 2004

Related U.S. Application Data
(63) Continuation of application No. PCT/JP02/12332, filed on Nov. 26, 2002.

(30) Foreign Application Priority Data
Nov. 28, 2001 (JP) 2001-362895
Nov. 28, 2001 (JP) 2001-362896
Nov. 28, 2001 (JP) 2001-362897
Dec. 25, 2001 (JP) 2001-392057

(51) Int. Cl. C07C 69/52; C07C 51/16

(52) U.S. Cl. 560/205; 562/545

(57) ABSTRACT

A first and second aspect of the invention relate to a process for producing a (meth)acrylic acid compound and a process for producing a (meth)acrylic ester, respectively. In particular, the second aspect of the invention relates to a process for (meth)acrylic ester production which includes a step in which by-products of a (meth)acrylic ester-yielding reaction are decomposed to recover a (meth)acrylic ester. A third aspect of the invention relates to a method of decomposing by-products of (meth)acrylic ester production in order to recover (meth)acrylic acid, a (meth)acrylic ester, and an alcohol through the decomposition of the by-products of (meth)acrylic ester production. A fourth and fifth aspect of the invention relate to a method of decomposing by-products of (meth)acrylic acid compound production in order to recover (meth)acrylic acid, a (meth)acrylic ester, and an alcohol through the decomposition of by-products of (meth)acrylic acid production and by-products of (meth)acrylic ester production.

### TECHNICAL FIELD

A first and second aspect of the invention relate to a process for producing a (meth)acrylic acid compound and a process for producing a (meth)acrylic ester, respectively. In particular, the second aspect of the invention relates to a process for (meth)acrylic ester production which includes a step in which by-products of a (meth)acrylic ester-yielding reaction are decomposed to recover a (meth)acrylic ester, etc.

A third aspect of the invention relates to a method of decomposing by-products of (meth)acrylic ester production in order to recover (meth)acrylic acid, a (meth)acrylic ester, an alcohol, etc. through the decomposition of the by-products of (meth)acrylic ester production.

A fourth and fifth aspect of the invention relate to a method of decomposing by-products of (meth)acrylic acid compound production in order to recover (meth)acrylic acid, a (meth)acrylic ester, an alcohol, etc. through the decomposition of by-products of (meth)acrylic acid production and by-products of (meth)acrylic ester production.

Incidentally, the term (meth)acrylic acid in this description is a general term for acrylic acid and methacrylic acid, and it may be either of these or may be both. Furthermore, the term (meth)acrylic acid compound is a general term for these acids and (meth)acrylic esters obtained from these acids and alcohols, and it means at least one of these.

### BACKGROUND ART

As is generally known, reactions for yielding acrylic acid to be used for producing an acrylic ester therefrom include the vapor-phase oxidation of propylene. Methods for this propylene oxidation for obtaining acrylic acid include a two-stage oxidation process in which oxidation to acrolein and subsequent oxidation to acrylic acid are conducted in separate reactors because these oxidation reactions differ in conditions and a process in which the starting material is oxidized directly to acrylic acid through one-stage oxidation.

FIG. 2 is an example of a flow diagram in which acrylic acid is yielded by two-stage oxidation and an acrylic ester is further yielded by reaction with an alcohol. Propylene, water vapor, and air pass through a first reactor and a second reactor which are packed with a molybdenum catalyst or the like, and the propylene is thus oxidized in two steps to give an acrylic-acid-containing gas. This acrylic-acid-containing gas is brought into contact with water in a condensation column (referred to also as collection column) to obtain an aqueous acrylic acid solution. An appropriate extractant is added to this solution. Extraction is conducted in an extraction column, and the extractant is separated in a solvent separation column. Subsequently, acetic acid is separated in an acetic acid separation column to obtain crude acrylic acid. By-products are separated from this crude acrylic acid in a rectifier, whereby purified acrylic acid is obtained. Furthermore, this acrylic acid (purified one) is esterified in an esterification column and then passes through an extraction column and a light-matter separation column to give a crude acrylic ester. By-products (high-boiling matters) are separated from this crude acrylic ester in a rectifier to give a purified acrylic ester.

Incidentally, there are cases where some kinds of acrylic esters are produced through the steps shown in FIG. 3. In this case, by-products are obtained as bottoms from an acrylic acid separation column or heavy-matter separation column.

In the acrylic ester production process shown in FIG. 3, acrylic acid, an alcohol, recovered acrylic acid, and a recovered alcohol each are fed to an esterification reactor. This esterification reactor is packed with a catalyst such as a strongly acidic ion-exchange resin. The esterification mixture taken out of this reactor, which comprises an ester yielded, unreacted acrylic acid, unreacted alcohol, water yielded, etc., is supplied to an acrylic acid separation column.

Bottoms containing unreacted acrylic acid are discharged through the bottom of this acrylic acid separation column and circulated to the esterification reactor. Part of the bottoms are supplied to a heavy-matter separation column, and heavy matters are separated through the bottom of the column, supplied to a high-boiling decomposition reactor (not shown), and decomposed. Decomposition products resulting from the decomposition, which include values, are circulated to the process. That part in the process to which the decomposition products are circulated varies depending on process conditions. High-boiling impurities such as polymers are removed outward from the high-boiling decomposition reactor.

The acrylic ester, unreacted alcohol, and water yielded are obtained as a distillate through the top of this acrylic acid separation column. Part of the distillate is circulated as a reflux to the acrylic acid separation column, while the remainder is supplied to an extraction column.

Water for alcohol extraction is supplied to this extraction column. The alcohol-containing water flowing out through the bottom of the column is supplied to an alcohol recovery column. The alcohol distilled is circulated to the esterification reactor.

The crude acrylic ester which has flowed out through the top of the extraction column is supplied to a low-boiling separation column. Low-boiling matters are discharged through the column top and circulated to a part in the process. That part in the process to which the low-boiling matters are circulated varies depending on process conditions. The crude acrylic ester from which low-boiling matters have been removed is supplied to an acrylic ester product purification column, and the acrylic ester having a high purity is obtained through the column top. The bottoms are circulated to a part in the process because they contain acrylic acid in a large amount. That part in the process to which the bottoms are circulated varies depending on process conditions.

In place of the solvent extraction method in which acrylic acid is recovered from the aqueous acrylic acid solution with an extractant, an azeotropic separation method is also employed recently which comprises distilling the solution-using water and an entrainer to obtain as a distillate an azeotropic mixture comprising water and the entrainer through the top of the azeotropic separation column and recover acrylic acid through the bottom of the column.

In the case of synthesizing a methacrylic ester, isobutylene or t-butyl alcohol is used in place of propylene. This starting material undergoes the same oxidation process and subsequent esterification process to give a purified methacrylic ester.

In the case of methacrylic acid and a methacrylic ester, isobutylene or t-butyl alcohol is used in place of propylene. The starting material undergoes the same oxidation process and subsequent esterification process to give purified methacrylic acid and a purified methacrylic ester.

Incidentally, for yielding a (meth)acrylic ester (acrylic ester or methacrylic ester), a method is also being employed in which the (meth)acrylic ester of a lower alcohol and a higher alcohol are subjected to transesterification using an acid or another substance as a catalyst to produce the (meth)acrylic ester of the higher alcohol. The crude (meth)acrylic ester obtained by this transesterification is subjected to steps such as catalyst separation, concentration, and purification to give a purified (meth)acrylic ester.

Fractions obtained by separating the crude acrylic acid, crude methacrylic acid, crude acrylic ester, and crude methacrylic ester through distillation/purification contain useful by-products including Michael addition products. These are hence decomposed to recover (meth)acrylic acid, esters thereof, the starting-material alcohol, etc.

Hitherto, the following have been used as methods for decomposing Michael addition products generated as by-products in acrylic acid or acrylic ester compound production. The pyrolysis method using no catalyst is generally employed in acrylic acid production processes (JP-A-11-012222), while a method in which the Michael addition products are decomposed by heating in the presence of a Lewis acid or Lewis base is generally employed in the case of acrylic ester production processes (JP-A-49-055614 and JP-A-09-110791). Furthermore, a reaction distillation technique in which a target decomposition reaction product is taken out by distillation while conducting a decomposition reaction is generally employed as a technique for the decomposition reaction of Michael addition products (JP-A-9-110791, JP-A-9-124551, and JP-A-8-225486). Also known is a method in which a Michael addition product generated as a by-product in an acrylic acid production step is pyrolyzed together with a Michael addition product generated as a by-product in an acrylic ester production step. Examples thereof include a method in which the Michael addition products are pyrolyzed by the reaction distillation technique without using a catalyst (JP-A-8-225486) and a method in which the Michael addition products are decomposed using a high-concentration acid catalyst (JP-A-9-183752).

In the method in which a Michael addition product generated as a by-product in a (meth)acrylic ester production step is subjected to a decomposition reaction using a Lewis acid or Lewis base as a catalyst to recover (meth)acrylic acid, a (meth)acrylic ester, and an alcohol, there are cases where use of decomposition reaction conditions suitable for obtaining a high recovery of the (meth)acrylic acid, (meth)acrylic ester, and alcohol results in decomposition products. which contain heavy matters in a high concentration and hence have an increased viscosity and reduced flowability to clog pipings.

As described above, Michael addition products generated as by-products in acrylic ester production steps have generally been treated by a method in which a decomposition reaction is conducted by the reaction distillation technique using a Lewis acid or Lewis base as a catalyst to recover acrylic acid, an acrylic ester, and an alcohol. In this method, however, when the recovery of effective ingredients such as acrylic acid, an alcohol, and an acrylic ester is heightened, then exceedingly heavy compounds accumulate in a high concentration on the bottom of the decomposition reaction distillation column and this has aroused troubles such as an increased viscosity and impaired flowability and, in an extreme case, clogging of a terminal piping. Furthermore, there has been a problem that the alcohol yielded by the decomposition reaction accelerates dehydration reactions by the action of the acid catalyst to generate an olefin or an ether. Such olefin and ether exert adverse influences such as the following. The olefin or ether makes it difficult to regulate the pressure in the reaction system or distillation system operated at a reduced pressure, or comes into a product acrylic ester to reduce its quality.

Furthermore, with respect to Michael addition products generated as by-products in acrylic acid production steps, a method is generally employed in which a pyrolysis reaction is conducted without using a catalyst to recover acrylic acid. However, this method also has had problems, for example, that to heighten the recovery of acrylic acid results in residues having reduced flowability to cause a clogging trouble to a terminal piping, as in the case of acrylic esters described above.

Moreover, the decomposition treatment in those two production processes is usually conducted in each production process, and each decomposition step has had problems such as the necessity of high-temperature operation and a high-grade material and the occurrence of a clogging trouble due to the impaired flowability of decomposition residues.

Consequently, there has been a desire for a process for decomposing Michael addition products, with respect to each of acrylic acid and an acrylic ester, which enables a high recovery to be stably obtained while eliminating those problems.

On the other hand, the method in which Michael addition products generated as by-products in (meth)acrylic ester production steps are subjected to a decomposition reaction using a Lewis acid or Lewis base as a catalyst to recover (meth)acrylic acid, a (meth)acrylic ester, and an alcohol has had problems, for example, that when decomposition reaction conditions suitable for obtaining a high recovery of the (meth)acrylic acid, (meth)acrylic ester, and alcohol are used, then an ether generates as a by-product in a considerably increased amount to contaminate the product, prevent a vacuum-system reactor or distillation column from being properly operated, or arouse other troubles.

Furthermore, the method in which Michael addition products generated as by-products in (meth)acrylic acid production steps are subjected to a pyrolysis reaction without using a catalyst to recover acrylic acid has had the following problems: a high-temperature operation is necessary; it is necessary to use a reaction vessel made of a high-grade material; the decomposition residues have poor flowability to arouse a clogging trouble or the like depending on operational fluctuations; and the like.

The problem of the generation of an ether as a by-product will be explained in detail using a process for producing the methyl ester of acrylic acid and a process for producing the n-butyl ester of acrylic acid as examples.

In the step of decomposing Michael addition products in the production of the methyl ester of acrylic acid, dimethyl ether generates as a by-product derived from methyl alcohol. Since this by-product dimethyl ether has a normal boiling point as extremely low as 248.3 K, it is less apt to condense in the decomposition reactor itself, a distillation column to which the recovered ether is to be sent, etc. Because of this, generation of the by-product in an increased amount arouses a trouble that it prevents the regulation of the vacuum system.

In the step of decomposing Michael addition products in the production of the n-butyl ester of acrylic acid, di-n-butyl ether generates as a by-product derived from n-butyl alcohol. When a fraction containing this di-n-butyl ether is recovered and sent to a reaction system or purification system, this poses a serious problem that since the normal boiling point of di-n-butyl ether is 413.4 K, which is exceedingly close to the normal boiling point of 420 K for n-butyl acrylate as a product, the ether contaminates the product.

Accordingly, an object of each of a first and second aspect of the invention is to overcome the existing problems described above and to provide a method in which by-products of a (meth)acrylic acid compound or (meth)acrylic ester production step, including a Michael addition reaction product, are pyrolyzed to recover a (meth)acrylic ester and which enables a high recovery to be stably obtained.

Furthermore, an object of a third aspect of the invention is to overcome the existing problems described above and to provide a method in which by-products of a (meth)acrylic ester production step, including a Michael addition reaction product, are decomposed using an acid as a catalyst to recover (meth)acrylic acid, a (meth)acrylic ester, and an alcohol and which, even under decomposition reaction conditions suitable for a high recovery, is effective in inhibiting the formation of by-product ethers problematic to the process.

Moreover, an object of a fourth aspect of the invention is to provide a method of decomposing by-products of (meth)acrylic acid compound production which is an effective and economical method in which Michael addition products yielded as by-products in (meth)acrylic acid and (meth)acrylic ester production steps are simultaneously decomposition-treated en bloc and the formation of by-product ethers and olefins in the step of decomposing the Michael addition products can be considerably diminished while stably maintaining a high recovery of (meth)acrylic acid, a (meth)acrylic ester, and an alcohol.

Furthermore, an object of a fifth aspect of the invention is to overcome the existing problems described above and to provide a method in which by-products of (meth)acrylic acid and (meth)acrylic ester production steps, including Michael addition reaction products, are decomposed using an acid as a catalyst to recover (meth)acrylic acid, a (meth)acrylic ester, and an alcohol and which, even under decomposition reaction conditions suitable for a high recovery, is effective in inhibiting the formation of by-product ethers problematic to the process and enables the Michael addition products yielded in the two steps to be simultaneously treated.

### DISCLOSURE OF THE INVENTION

The process for (meth)acrylic acid compound production according to the first aspect of the invention is a process which has both (meth)acrylic acid production facilities and (meth)acrylic ester production facilities and in which by-products taken out of a purification step for purifying a reaction mixture of a (meth)acrylic ester are pyrolyzed to recover the (meth)acrylic ester therefrom, characterized in that the pyrolysis reaction of the by-products is conducted substantially in a liquid phase and at least part of the products of the pyrolysis reaction are returned to a (meth)acrylic ester purification step.

Furthermore, the process for (meth)acrylic ester production according to the second aspect of the invention is a process for producing a (meth)acrylic ester which comprises a (meth)acrylic ester-yielding reaction step and a step in which by-products separated from the step of yielding are pyrolyzed to recover (meth)acrylic acid, a (meth)acrylic ester, and an alcohol therefrom, and is characterized in that the pyrolysis reaction is conducted substantially in a liquid phase and at least 50% of the products of the pyrolysis reaction are returned to an upstream step.

When the pyrolysis of by-products including Michael addition products is conducted in a liquid phase and at least 50% of the products of this pyrolysis reaction are recycled, as in the second aspect of the invention, then a (meth)acrylic ester can be recovered at a high recovery and process clogging or the like is prevented.

Moreover, the method of decomposing by-products of (meth)acrylic ester production according to the third aspect of the invention comprises decomposing the by-products of (meth)acrylic ester production in the presence of an acid catalyst, and is characterized in that the acid catalyst is added in an amount of from 0.1 to 1.0% by weight based on the by-products.

In the step of decomposing a Michael addition product generated as a by-product in a (meth)acrylic ester production step, a large amount of an acid catalyst has hitherto been used in order to heighten the recovery. However, the use of a large amount of the catalyst has had a drawback that an ether generates as a by-product due to, e.g., the dehydrating dimerization reaction of an alcohol and the ether which has generated here may prevent the regulation of the vacuum system or contaminate the product, as stated above.

As a result of investigations made by the present inventors, it has been found that to reduce rather than increase the amount of a catalyst to be used is effective in diminishing ether generation and improving productivity.

Moreover, according to the third aspect of the invention, which is based on that finding, Michael addition products can be efficiently decomposed.

Furthermore, the method of decomposing by-products of (meth)acrylic acid compound production according to the fourth aspect of the invention comprises pyrolyzing a mixture of by-products of (meth)acrylic acid production and by-products of (meth)acrylic ester production in a liquid phase, and is characterized in that at least 50% of the products of the pyrolysis reaction are returned to a (meth)acrylic ester production step.

According to the fourth aspect of the invention, by-products of (meth)acrylic ester production, i.e., bottoms from a rectifier which contain in a high concentration a Michael addition product generated as a by-product of (meth)acrylic ester production, are decomposed together with by-products of (meth)acrylic acid production, i.e., bottoms from a rectifier which contain in a high concentration a Michael addition product generated as a by-product of (meth)acrylic acid production, not by a reaction distillation technique but by a pyrolysis reaction while maintaining a liquid phase. In addition, a large proportion of the pyrolysis reaction products are recycled to a (meth)acrylic ester production step. Due to this constitution and especially when the pyrolysis reaction is conducted without using a catalyst, the recovery of (meth)acrylic acid, a (meth)acrylic ester, and an alcohol can be heightened and a stable continuous operation is possible over long while inhibiting the generation of an ether or olefin as a by-product.

One of the advantages of the fourth aspect of the invention resides in that the decomposition reactors which are required to be made of a high-grade material and which in related-art techniques have had troubles such as clogging and have been required to be installed respectively in a (meth)acrylic acid production step and a (meth)acrylic ester production step can be united into one, which may be installed in a (meth)acrylic ester production step only, and that values obtained by the decomposition can be recovered within the (meth)acrylic ester production step at a high recovery. Thus, the construction cost, labor cost, and utility cost can be considerably reduced, and a substantial cost reduction can be attained.

Another great advantage of the fourth aspect of the invention resides in that although by-products of (meth)acrylic ester production have hitherto been decomposed using an acid catalyst, the method according to the fourth aspect of the invention is effective in preventing the generation of an ether or olefin as an alcohol-derived by-product, which is problematic to decomposition reactions with an acid catalyst. The reasons for this are as follows.

(1) Since the decomposition reaction can be efficiently conducted without using a catalyst, the progress of the dehydration reaction catalyzed by an acid catalyst is retarded and the generation of an ether or olefin as an alcohol-derived by-product is inhibited.

(2) By-products of (meth)acrylic acid production contain no alcohol-derived compound. Because of this, the simultaneous treatment of by-products of (meth)acrylic acid production produces a diluting effect, which lowers the alcohol concentration and thereby inhibits the dehydration reaction.

(3) The simultaneous treatment of by-products of (meth)acrylic acid production gives decomposition products having an increased (meth)acrylic acid concentration. Consequently, the alcohol yielded by the decomposition undergoes esterification with (meth)acrylic acid and is stabilized, without undergoing a dehydration reaction. Thus, the generation of an ether or olefin as an alcohol-derived by-product is inhibited.

Although (meth)acrylic acid not only undergoes the Michael addition reaction but also is apt to undergo a radical polymerization reaction, the concentration of (meth)acrylic acid is lowered by treating by-products of (meth)acrylic acid production and by-products of (meth)acrylic ester production en bloc according to the fourth aspect of the invention. This method thus produces an additional effect that the undesirable polymerization reaction during the pyrolysis reaction conducted at a high temperature is inhibited.

The fourth aspect of the invention further has an advantage that since by-products of (meth)acrylic acid production and by-products of (meth)acrylic ester production are treated en bloc, the amount of the decomposition residues flowing per unit hour increases and the residues can have improved flowability in a residue discharge piping.

The fourth aspect of the invention is especially preferably conducted in the following manner. Bottoms from a rectifier which include a Michael addition reaction product generated as a by-product in a (meth)acrylic ester production step are concentrated with a film evaporator. A Michael addition product generated as a by-product in a (meth)acrylic acid production step is added to the resultant concentrate to prepare a feed material. A pyrolysis reaction is conducted for from 0.5 to 50 hours preferably at from 120 to 280° C. under such conditions that a liquid phase is substantially maintained. Preferably at least 80% of the resultant pyrolysis reaction products are recycled to the (meth)acrylic ester production step, preferably to the film evaporator as a reboiler for the rectifier.

Furthermore, the method of decomposing by-products of (meth)acrylic acid compound production according to the fifth aspect of the invention comprises decomposing a mixture of by-products of (meth)acrylic acid production and by-products of (meth)acrylic ester production in the presence of an acid catalyst, and is characterized in that the acid catalyst is added in an amount of from 0.1 to 1.0% by weight based on the mixture.

In the step of decomposing a Michael addition product generated as a by-product in an acrylic ester production step, a large amount of an acid catalyst has hitherto been used in order to heighten the recovery. However, the use of a large amount of the catalyst has had a drawback that an ether generates as a by-product due to, e.g., the dehydrating dimerization reaction of an alcohol and the ether which has generated here may prevent the regulation of the vacuum system or contaminate the product, as stated above.

As a result of investigations made by the present inventors, it has been found that to reduce rather than increase the amount of a catalyst to be used is effective in diminishing ether generation and improving productivity.

In the fifth aspect of the invention, since a Michael addition product generated as a by-product in a (meth)acrylic acid production step and a Michael addition product generated as a by-product in a (meth)acrylic ester production step are decomposed en bloc, the decomposition of the Michael addition products can be efficiently conducted.

### BRIEF DESCRIPTION OF THE DRAWINGS

FIG. 1 is a flow diagram of acrylic ester production according to the second aspect of the invention.

FIG. 2 is one example of flow diagrams of the production of acrylic acid and an acrylic ester.

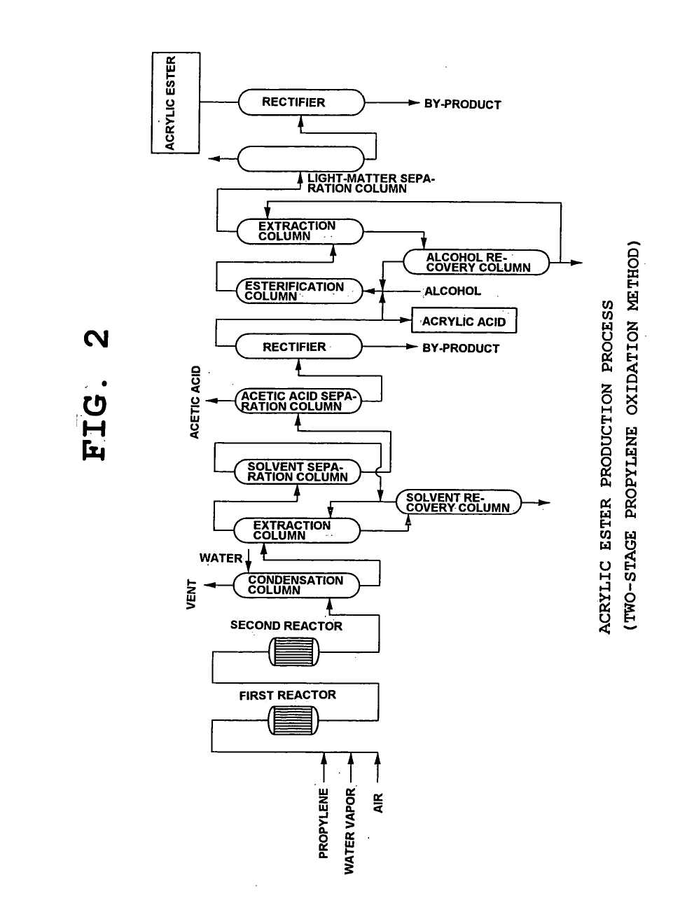

FIG. 3 is another example of flow diagrams of the production of an acrylic ester.

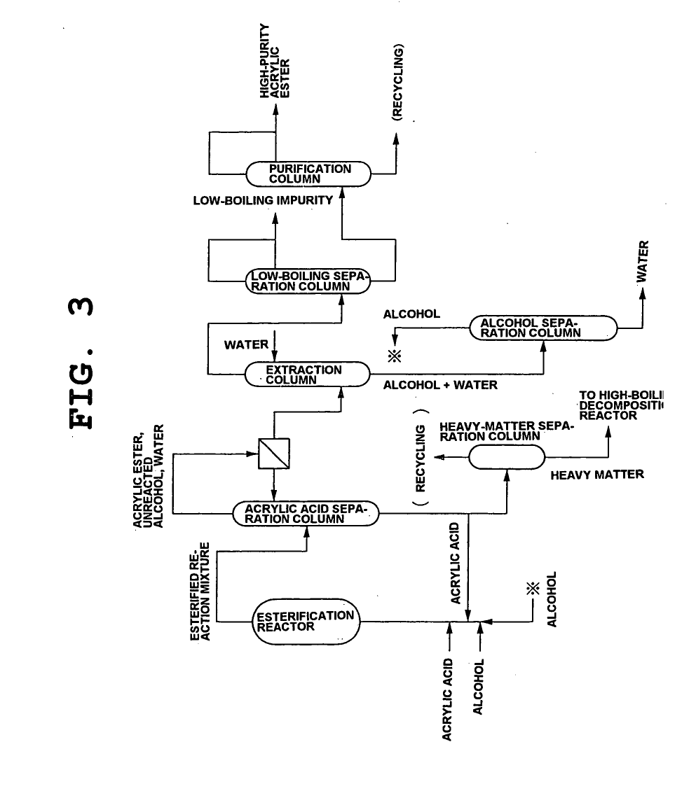

### BEST MODE FOR CARRYING OUT THE INVENTION

The first and second aspects of the invention will be explained below in more detail.

The process for (meth)acrylic acid compound production according to the first aspect of the invention may be characterized in that at least 50% of the products of the pyrolysis reaction are returned to a purification step.

The process for (meth)acrylic acid compound production according to the first aspect of the invention may be characterized in that the by-products are bottoms from a distillation column for separating heavy matters in a (meth)acrylic ester purification step.

The process for (meth)acrylic acid compound production according to the first aspect of the invention may be characterized in that the pyrolysis reaction of the by-products is conducted in the presence of an acid catalyst and the acid catalyst is added in an amount of from 0.1 to 1.0% by weight based on the by-products.

The process for (meth)acrylic acid compound production according to the first aspect of the invention may be characterized in that the by-products. to be subjected to pyrolysis reaction are a mixture of by-products of (meth)acrylic acid production and by-products of (meth)acrylic ester production.

The process for (meth)acrylic acid compound production according to the first aspect of the invention may be characterized in that the by-products of (meth)acrylic acid production are bottoms from a rectifier for separating heavy matters in a (meth)acrylic acid purification step and the by-products of (meth)acrylic ester production are bottoms from a rectifier for separating heavy matters in a (meth)acrylic ester purification step.

The process for (meth)acrylic acid compound production according to the first aspect of the invention may be characterized in that the mixture of by-products of (meth)acrylic acid production and by-products of (meth)acrylic ester production is pyrolyzed in the presence of an acid catalyst and the acid catalyst is added in an amount of from 0.1 to 1.0% by weight based on the mixture.

The process for (meth)acrylic acid compound production according to the first aspect of the invention may be characterized in that the rectifier for separating heavy matters in a (meth)acrylic ester purification step is equipped with a film evaporator as a reboiler.

The process for (meth)acrylic acid compound production according to the first aspect of the invention may be characterized in that at least 80% of the pyrolysis reaction products are returned to a (meth)acrylic ester purification step.

The process for (meth)acrylic acid compound production according to the first aspect of the invention may be characterized in that the temperature for the pyrolysis reaction is from 120 to 280° C. and the time period of the pyrolysis reaction is from 0.5 to 50 hours.

The (meth)acrylic ester in the second aspect of the invention is not particularly limited. However, examples thereof include methyl (meth)acrylate, ethyl (meth)acrylate, n-butyl (meth)acrylate, isobutyl (meth)acrylate, 2-ethylhexyl (meth)acrylate, isononyl (meth)acrylate, isodecyl (meth)acrylate, and the like.

The Michael addition product in the second aspect of the invention is a by-product which generates in a reaction step or purification step in producing (meth)acrylic esters, and is a compound formed by the Michael addition of (meth)acrylic acid, an alcohol, or water to a compound having a (meth)acryloyl group which is present during the production of these esters. Examples of the compound having a (meth)acryloyl group which is present during the production include the (meth)acrylic acid used as a starting material, (meth)acrylic esters, carboxylic acids having an acryloyl group, such as the β-acryloxypropionic acid or β-methacryloxyisobutyric acid (hereinafter, dimer) formed by the Michael addition of the (meth)acrylic acid to itself, a (meth)acrylic acid trimer (hereinafter, trimer) formed by the Michael addition of (meth)acrylic acid to the dimer, and a (meth)acrylic acid tetramer (hereinafter, tetramer) formed by the Michael addition of (meth)acrylic acid to the trimer, and the corresponding (meth)acrylic esters formed by esterifying these carboxylic acids having a (meth)acryloyl group with alcohols. Specific examples of the Michael addition product in the invention include β-acryloxypropionic acid, β-methacryloxyisobutyric acid, and esters of these; β-alkoxypropionic acids or β-alkoxyisobutyric acids and esters of these; β-hydroxypropionic acid or isobutyric acid and esters of these; the dimers, trimers, tetramers, etc. of these acids; esters of these; β-acryloxy-substituted forms and β-hydroxy-substituted forms thereof; and the like.

In the second aspect of the invention, the by-products of the (meth)acrylic ester-yielding reaction preferably include a Michael addition product formed by the addition of water, methanol, ethanol, butanol, or (meth)acrylic acid to the α-position or β-position of a (meth)acryloyl group.

In the second aspect of the invention, the (meth)acrylic acid to be used for producing a (meth)acrylic ester therefrom preferably is one obtained by the catalytic vapor-phase oxidation of propane, propylene, acrolein, isobutylene, t-butyl alcohol, or the like. A gaseous oxidation reaction product is rapidly cooled and quenched with water. Thereafter, water/acrylic acid separation is conducted by the azeotropic distillation method using an entrainer or by the extraction method using a solvent. Furthermore, low-boiling compounds including acetic acid are separated and, thereafter, heavy matters including Michael addition products are separated to thereby produce high-purity (meth)acrylic acid. Incidentally, water and acetic acid may be simultaneously separated with an entrainer.

For producing a (meth)acrylic ester in the second aspect of the invention, use may be made of either a method in which (meth)acrylic acid is esterified with an alcohol or a method in which the acrylic ester of a lower alcohol is subjected to transesterification with a higher alcohol to produce the acrylic ester of the higher alcohol. The production process may be either a batch process or a continuous process. An acid catalyst is generally used as a catalyst for the esterification or transesterification.

The (meth)acrylic ester production process according to the second aspect of the invention preferably comprises a reaction step and a purification step in which the crude acrylic ester solution obtained in the reaction step is subjected to cleaning, extraction, evaporation, distillation, etc. in order to conduct catalyst separation, concentration/purification, etc. Conditions in the reaction step, such as the molar ratio between starting materials, i.e., (meth)acrylic acid or a (meth)acrylic ester and an alcohol, the kind and amount of a catalyst to be used for the reaction, reaction mode, and reaction conditions, are suitably selected according to the kind of the alcohol to be used as a starting material.

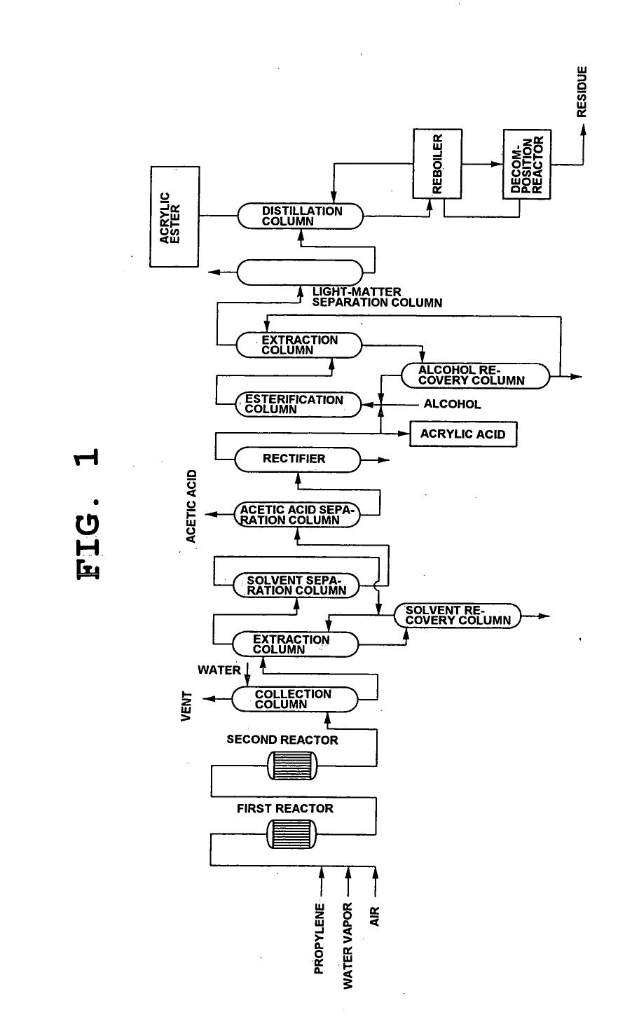

The Michael addition product which generates as a major by-product in the esterification step in the second aspect of the invention accumulates as a heavy matter in a high concentration on the bottom of a distillation column (in the case of FIG. 1, the acrylic ester rectifier) for recovering an effective ingredient. Although the bottoms contain the Michael addition product described above in a high concentration, they further contain acrylic acid and/or an acrylic ester in a considerable amount. Furthermore, the bottoms contain heavy matters such as the polymerization inhibitor used in the process, oligomers and polymers generated in the process, and high-boiling impurities contained in the starting materials or reaction products derived from these. There also are cases where the bottoms contain the catalyst used in the esterification or transesterification step.

The bottoms are decomposed by heating in the presence of a Lewis acid or Lewis base, and effective ingredients obtained are recovered and sent to the reaction step or purification step. The distillation column for separating heavy matters may be any of a distillation column for separating acrylic acid from heavy matters, a distillation column for separating an acrylic ester from heavy matters, a distillation column for separating acrylic acid, an alcohol, and an acrylic ester from heavy matters, and the like.

The distillation column is preferably equipped with a reboiler. This reboiler preferably is a film evaporator because the bottoms have high viscosity and polymerizability. The type of the film evaporator is not particularly limited. Incidentally, the distillation column may be equipped with a reboiler of the thermosyphon type, forced circulation type, or the like, and a film evaporator may be used as an aid to any of these.

In the second aspect of the invention, the decomposition reaction of the Michael addition product can be conducted by any of the continuous process, batch process, semi-batch process, intermittent-discharge process, and the like. However, the continuous process is preferred. The type of the reactor also is not particularly limited, and a reactor of any type can be employed, such as a flow-through type tubular reactor, complete mixing type stirring-vessel reactor, circulating complete-mixing vessel reactor, reactor having a mere cavity, or the like.

The pyrolysis reaction of the Michael addition product is conducted not by the reaction distillation technique but under such conditions that a liquid phase is substantially maintained. Although a catalyst is not always necessary for the pyrolysis, a Lewis acid or Lewis base catalyst can be used.

The temperature for the decomposition reaction is preferably from 120 to 280° C., especially from 140 to 240° C. The liquid residence time as calculated from the amount of the liquid discharged is preferably from 0.5 to 50 hours, especially from 1 to 10 hours. In the case where the decomposition reaction is conducted continuously, the liquid residence time calculated from the amount of the liquid discharged can be regarded as the reaction time. For example, when the liquid capacity of the reactor is 500 L and the liquid discharge amount is 100 L/H, then the residence time is 5 hours.

In the second aspect of the invention, most of the pyrolysis reaction products are recycled. Part of the remainder is discharged as a decomposition residue, giving a waste or fuel. Although the place to which the pyrolysis reaction products are recycled is not particularly limited, it is preferred to feed the products to the bottom of a heavy-matter separation column or to the film evaporator which is a reboiler for the heavy-matter separation column. Higher recycling proportions are preferred because the amount of the residue to be discharged decreases. Specifically, it is preferred to recycle at least 80% of the pyrolysis reaction products. As the recycling proportion increases, the recovery becomes higher and the residue comes to have a higher viscosity and poorer flowability. Consequently, the upper limit thereof is suitably selected in a range in which continuous operation is possible.

The third aspect of the invention will be explained below in more detail.

The (meth)acrylic ester in the third aspect of the invention is not particularly limited. However, (meth)acrylic esters produced from starting-material alcohols having no branch are preferred, such as methyl (meth)acrylate, ethyl (meth)acrylate, n-butyl (meth)acrylate, n-hexyl (meth)acrylate, n-octyl (meth)acrylate, and methoxyethyl (meth)acrylate. Most preferred of these is n-butyl (meth)acrylate.

The Michael addition product is a by-product which generates in a reaction step or purification step in producing (meth)acrylic esters, and is a compound formed by the Michael addition of (meth)acrylic acid, an alcohol, or water to a compound having a (meth)acryloyl group which is present during the production of these esters. Examples of the compound having a (meth)acryloyl group which is present during the production include (meth)acrylic acid, carboxylic acids such as the β-acryloxypropionic acid or β-methacryloxyisobutyric acid (hereinafter, dimer) formed by the Michael addition of the (meth)acrylic acid to itself, a (meth)acrylic acid trimer (hereinafter, trimer) formed by the Michael addition of (meth)acrylic acid to the dimer, and a (meth)acrylic acid tetramer (hereinafter, tetramer), and the corresponding (meth)acrylic esters formed by esterifying these carboxylic acids having a (meth)acryloyl group with alcohols. Specific examples of the Michael addition product in the third aspect of the invention include β-acryloxypropionic acid, β-methacryloxyisobutyric acid, and esters of these; β-alkoxypropionic acids or β-alkoxyisobutyric acids and esters of these; β-hydroxypropionic acid or isobutyric acid and esters and aldehydes of these; the dimers, trimers, tetramers, etc. of these acids; esters of these; β-acryloxy-substituted forms, β-alkoxy-substituted forms, and β-hydroxy-substituted forms thereof; and the like.

In the third aspect of the invention, the by-products of the (meth)acrylic ester production preferably include a Michael addition product formed by the addition of water, methanol, ethanol, n-butanol, or (meth)acrylic acid to the α-position or β-position of a (meth)acryloyl group.

In the third aspect of the invention, the (meth)acrylic acid to be used for producing a (meth)acrylic ester therefrom may be produced by the same method as in the second aspect of the invention.

Methods usable for producing a (meth)acrylic ester in the third aspect of the invention are the same as the methods in the second aspect of the invention.

The (meth)acrylic ester production process according to the third aspect of the invention may be the same as the process according to the second aspect of the invention.

The Michael addition product which generates as a major by-product in the esterification step accumulates as a heavy matter in a high concentration on the bottom of a distillation column for recovering an effective ingredient.

In the third aspect of the invention, the decomposition reaction of the Michael addition product can be conducted by any of the continuous process, batch process, semi-batch process, intermittent-discharge process, and the like. However, the continuous process is preferred. The type of the reactor also is not particularly limited, and a reactor of any type can be employed, such as a flow-through type tubular reactor, thin-film flowing-down type reactor, complete mixing type stirring vessel reactor, circulating complete-mixing vessel reactor, or the like. For recovering a useful ingredient contained in the products of the decomposition reaction, use can be made of either a method in which the useful ingredient is obtained by evaporation or distillation during the reaction or a method in which after the decomposition reaction, the useful ingredient is obtained by evaporation or distillation. However, the former method, which is a reaction distillation technique, is preferred for obtaining a high recovery.

In the case where the reaction distillation technique is employed, the reaction pressure considerably depends on the reaction temperature which will be described later. A pressure is employed at which most of the useful ingredients which have been yielded by the decomposition reaction and of the useful ingredients which were contained in the feed material for the decomposition reaction, such as acrylic acid, an acrylic ester, and an alcohol, vaporize.

The catalyst is selected from inorganic acids such as sulfuric acid and phosphoric acid, organic acids such as methanesulfonic acid and p-toluenesulfonic acid, and the like. However, organic acids are preferred.

In the third aspect of the invention, the concentration of the acid catalyst is from 0.1 to 1.0% by weight, preferably from 0.2 to 0.8% by weight, based on the feed liquid.

The temperature for the decomposition reaction is preferably from 120 to 200° C. The liquid residence time as calculated from the amount of the liquid discharged is preferably from 0.5 to 50 hours, especially from 2 to 20 hours. In the case where the decomposition reaction is conducted continuously, the liquid residence time calculated from the amount of the liquid discharged can be regarded as the reaction time. For example, when the liquid capacity of the reactor is 500 L and the liquid discharge amount is 100 L/H, then the residence time is 5 hours.

Incidentally, the ordinary decomposition reaction conditions which have hitherto been employed include a p-toluenesulfonic acid concentration of from 5 to 15% by weight based on the feed liquid, a decomposition reaction temperature of from 180 to 230° C., and a reaction time of from 0.1 to 4.0 hours. The present inventors analyzed reactions yielding an ether as a by-product and decomposition reactions of Michael addition products from many angles, and have found that to use an acid as a catalyst in a low concentration and to employ a relatively low decomposition temperature are preferred for inhibiting the generation of ethers as by-products.

When the decomposition reaction conditions according to the third aspect of the invention are employed, the progress of the decomposition reaction of Michael addition products becomes slightly slow. However, a sufficiently high recovery is obtained when the reaction time is prolonged to some degree.

Incidentally, as a result of various experiments, it was found that the decomposition residue obtained under the decomposition reaction conditions according to the third aspect of the invention has a lower viscosity and better flowability than decomposition residues obtained under ordinary decomposition reaction conditions.

Embodiments of the method of decomposing by-products of (meth)acrylic acid compound production according to the fourth aspect of the invention will be explained below in detail. Hereinafter, the term (meth)acrolein indicates either or both of acrolein and methacrolein.

The (meth)acrylic acid in the fourth aspect of the invention preferably is one obtained by the catalytic vapor-phase oxidation of propane, propylene, acrolein, isobutylene, t-butyl alcohol, or the like. A gaseous oxidation reaction product is rapidly cooled and quenched with water. Thereafter, water/(meth)acrylic acid separation is conducted by the azeotropic distillation method using an entrainer or by the extraction method using a solvent. Furthermore, low-boiling compounds including acetic acid are separated and, thereafter, heavy matters including Michael addition products are separated to thereby produce high-purity (meth)acrylic acid. Incidentally, water and acetic acid may be simultaneously separated with an entrainer. Since the Michael addition products accumulate in heavy matters in a high concentration, it is preferred to mix this fraction, usually the bottoms from a rectifier, with by-products of (meth)acrylic ester production and treat the resultant mixture en bloc.

The (meth)acrylic ester in the fourth aspect of the invention is not particularly limited, and examples thereof include methyl (meth)acrylate, ethyl (meth)acrylate, n-butyl (meth)acrylate, isobutyl (meth)acrylate, n-hexyl (meth)acrylate, 2-ethylhexyl (meth)acrylate, n-octyl (meth)acrylate, methoxyethyl (meth)acrylate, isononyl (meth)acrylate, isodecyl (meth)acrylate, and the like. However, (meth)acrylic esters produced from raw-material alcohols having no branch are especially preferred. Especially preferred of these are methyl (meth)acrylate, ethyl (meth)acrylate, and n-butyl (meth)acrylate.

The Michael addition product is a by-product which generates in a reaction step or purification step in producing (meth)acrylic acid and (meth)acrylic esters, and is a compound formed by the Michael addition of (meth)acrylic acid, acetic acid, an alcohol, or water to a compound having a (meth)acryloyl group which is present during the production of the acid or esters. Examples of the compound having a (meth)acryloyl group which is present during the production include (meth)acrolein, (meth)acrylic acid, carboxylic acids having a (meth)acryloyl group, such as the β-acryloxypropionic acid or β-methacryloxyisobutyric acid (hereinafter, both are inclusively referred to as dimer) formed by the Michael addition of the (meth)acrylic acid to itself, a (meth)acrylic acid trimer (hereinafter, trimer) formed by the Michael addition of (meth)acrylic acid to the dimer, and a (meth)acrylic acid tetramer (hereinafter, tetramer) formed by the Michael addition of (meth)acrylic acid to the trimer, and the corresponding (meth)acrylic esters formed by esterifying these carboxylic acids having a (meth)acryloyl group with alcohols. Examples thereof further include compounds likewise formed by the Michael addition of (meth)acrylic acid to (meth)acrolein. Specific examples of the Michael addition product in the fourth aspect of the invention include β-acryloxypropionic acid or β-methacryloxyisobutyric acid and esters and aldehydes of these (β-acryloxypropanol or β-methacryloxyisobutanol); β-alkoxypropionic acids and esters of these; β-hydroxypropionic acid or β-hydroxyisobutyric acid and esters and aldehydes of these; the dimers, trimers, tetramers, etc. of these acids; esters of these; β-acryloxy-substituted forms, β-acetoxy-substituted forms, β-alkoxy-substituted forms, and β-hydroxy-substituted forms thereof; and the like. Incidentally, compounds formed by the Michael addition of acetic acid to a (meth)acryloyl group are also present although the amount thereof is slight.

In the fourth aspect of the invention, a mixture of the by-products of (meth)acrylic acid production and the by-products of (meth)acrylic ester production preferably contains a Michael addition product formed by the addition of water, an alcohol, or (meth)acrylic acid to the α-position or β-position of a (meth)acryloyl group.

Methods usable for producing a (meth)acrylic ester in the fourth aspect of the invention are the same as the methods in the second aspect of the invention.

The (meth)acrylic ester production process preferably comprises a reaction step and a purification step in which the crude (meth)acrylic ester solution obtained in the reaction step is subjected to cleaning, extraction, evaporation, distillation, etc. as unit operations for conducting catalyst separation, concentration/purification, etc. Conditions in the reaction step, such as the molar ratio between starting materials, i.e., (meth)acrylic acid or a (meth)acrylic ester and an alcohol, the kind and amount of a catalyst to be used for the reaction, reaction mode, and reaction conditions, are suitably selected according to the kind of the alcohol to be used as a starting material.

The Michael addition product which generates as a major by-product in the reaction accumulates in a high concentration on the bottom of a distillation column (rectifier) for separating heavy matters. Consequently, in the fourth aspect of the invention, the bottoms are treated by pyrolyzing them together with by-products sent from the preceding step of (meth)acrylic acid production. Effective ingredients obtained are recovered and sent to the reaction step or purification step for the (meth)acrylic ester.

Incidentally, the distillation column for separating heavy matters varies depending on the process employed and the kind of the (meth)acrylic ester to be produced. In general, such distillation columns include one for separating (meth)acrylic acid from heavy matters, one for separating a (meth)acrylic ester from heavy matters, and one for separating (meth)acrylic acid, an alcohol, and a (meth)acrylic ester from heavy matters. However, the fourth aspect of the invention can be applied to all of these.

The distillation column for separating heavy matters (rectifier; hereinafter sometimes referred to as “heavy-matter separation column”) in the step of (meth)acrylic ester production in the fourth aspect of the invention may be equipped with a reboiler of the thermosyphon type, forced circulation type, or the like. However, a film evaporator may be used as an aid to any of these. More preferred is a rectifier employing a film evaporator as the only reboiler. The type of the film evaporator is not particularly limited. The reason why a film evaporator is preferred as a reboiler for the rectifier is that the bottoms from the heavy-matter separation column have high viscosity and polymerizability.

Although the bottoms from the heavy-matter separation column contain the Michael addition product described above in a high concentration, they further contain (meth)acrylic acid and/or a (meth)acrylic ester in a considerable amount. Furthermore, the bottoms contain heavy matters such as the polymerization inhibitor used in the process, oligomers and polymers generated in the process, and high-boiling impurities contained in the starting materials or reaction products derived from these. There also are cases where the bottoms contain the catalyst used in the esterification or transesterification step. However, bottoms containing no acid catalyst are preferred from the standpoint of inhibiting the generation of an olefin or ether as a by-product during the decomposition reaction.

As stated above, the Michael addition product which has generated in the step of (meth)acrylic acid production usually accumulates in a high concentration on the bottom of a distillation column (rectifier) for separating the (meth)acrylic acid product from heavy matters. The bottoms further contain (meth)acrylic acid in a considerable amount, and furthermore contain the polymerization inhibitor used in the process and oligomers and heavy metals generated in the process.

In the fourth aspect of the invention, the pyrolysis reaction of the mixture of by-products of (meth)acrylic acid production, which include a Michael addition product, and by-products of (meth)acrylic ester production can be conducted by any technique such as the continuous process, batch process, semi-batch process, or intermittent-discharge process. However, the continuous process is preferred. The type of the reactor also is not particularly limited, and any reactor can be employed, such as a flow-through type tubular reactor, complete mixing vessel type stirring reactor, circulating complete-mixing vessel reactor, or reactor having a mere cavity.

The pyrolysis reaction in the fourth aspect of the invention is conducted not by the reaction distillation technique but under such conditions that a liquid phase is substantially maintained. Although known Lewis acid or Lewis base catalysts may be used, it is preferred to use no catalyst because use of these catalysts results in the generation of an alcohol-derived ether or olefin.

Conditions for the decomposition reaction preferably include a temperature of from 120 to 280° C., preferably from 140 to 240° C., and a liquid residence time as calculated from liquid discharge amount of from 0.5 to 50 hours, preferably from 1 to 20 hours.

The fourth aspect of the invention is characterized in that at least 50% of the products of the pyrolysis reaction are returned to the step of (meth)acrylic ester production. The remainder of the pyrolysis reaction products is discharged as a decomposition residue, giving a waste or fuel. The place to which the pyrolysis reaction products are returned is not particularly limited as long as it is in the step of (meth)acrylic ester production. It is, however, preferred to feed the products to the bottom of a heavy-matter separation column or to the film evaporator which is a reboiler for the heavy-matter separation column. By thus returning the pyrolysis reaction products to the step of (meth)acrylic ester production, most of the (meth)acrylic acid, (meth)acrylic ester, and alcohol which are values contained in the pyrolysis reaction products can be taken out as a distillate from the heavy-matter separation column, circulated to the reaction step or purification step for the (meth)acrylic ester, and recovered. The proportion of the pyrolysis reaction products to be returned to the step of (meth)acrylic ester production to all pyrolysis reaction products (hereinafter sometimes referred to as “recycling proportion”) preferably is higher because higher proportions thereof result in smaller amounts of the residue to be discharged. In the fourth aspect of the invention, at least 50%, preferably at least 80%, of the pyrolysis reaction products are returned to the step of (meth)acrylic ester production. The higher the recycling proportion, the higher the recovery. However, higher recycling proportions result in a residue having an increased viscosity and impaired flowability. The upper limit of the recycling proportion is hence suitably selected in a range in which continuous operation is possible. In general, however, the recycling proportion is 95% or lower.

The fifth aspect of the invention will be explained below in more detail. Hereinafter, the term (meth)acrolein indicates either or both of acrolein and methacrolein.

The (meth)acrylic ester in the fifth aspect of the invention is not particularly limited. However, (meth)acrylic esters produced from raw-material alcohols having no branch are preferred, such as methyl (meth)acrylate, ethyl (meth)acrylate, n-butyl (meth)acrylate, n-hexyl (meth)acrylate, n-octyl (meth)acrylate, and methoxyethyl (meth)acrylate. Most Preferred of these is n-butyl (meth)acrylate.

The Michael addition product in the fifth aspect of the invention is a by-product which generates in a reaction step or purification step in producing (meth)acrylic acid and (meth)acrylic esters, and is a compound formed by the Michael addition of (meth)acrylic acid, acetic acid, an alcohol, or water to a compound having a (meth)acryloyl group which is present during the production of the acid or esters. Examples of the compound having a (meth)acryloyl group which is present during the production include (meth)acrolein, (meth)acrylic acid, carboxylic acids such as the β-acryloxypropionic acid or β-methacryloxyisobutyric acid (hereinafter, both are inclusively referred to as dimer) formed by the Michael addition of the (meth)acrylic acid to itself, a (meth)acrylic acid trimer (hereinafter, trimer) formed by the Michael addition of (meth)acrylic acid to the dimer, and a (meth)acrylic acid tetramer (hereinafter, tetramer), and the corresponding (meth)acrylic esters formed by esterifying these carboxylic acids having a (meth)acryloyl group with alcohols. Examples thereof further include compounds likewise formed by the Michael addition of (meth)acrylic acid to (meth)acrolein. Specific examples of the Michael addition product in the fifth aspect of the invention include β-acryloxypropionic acid or β-methacryloxyisobutyric acid and esters and aldehydes of these (β-acryloxypropanal or β-methacryloxyisobutanal); β-alkoxypropionic acids and esters of these; β-hydroxypropionic acid or β-hydroxyisobutyric acid and esters and aldehydes of these; the dimers, trimers, tetramers, etc. of these acids; esters of these; β-acryloxy-substituted forms, β-acetoxy-substituted forms, β-alkoxy-substituted forms, and β-hydroxy-substituted forms thereof; and the like.

In the fifth aspect of the invention, a mixture of the by-products of (meth)acrylic acid production and the by-products of (meth)acrylic ester production preferably contains a Michael addition product formed by the addition of water, an alcohol, or (meth)acrylic acid to the α-position or β-position of a (meth)acryloyl group.

The (meth)acrylic acid in the fifth aspect of the invention preferably is one obtained by the catalytic vapor-phase oxidation of propane, propylene, acrolein, isobutylene, t-butyl alcohol, or the like. A gaseous oxidation reaction product is rapidly cooled and quenched with water. Thereafter, water/(meth)acrylic acid separation is conducted by the azeotropic distillation method using an entrainer or by the extraction method using a solvent. Furthermore, low-boiling compounds including acetic acid are separated and, thereafter, heavy matters including Michael addition products are separated to thereby produce high-purity (meth)acrylic acid. Incidentally, water and acetic acid may be simultaneously separated with an entrainer. The Michael addition products accumulate in heavy matters in a high concentration.

Methods usable for producing a (meth)acrylic ester in the fifth aspect of the invention are the same as the methods in the second aspect of the invention.

The (meth)acrylic ester production process according to the fifth aspect of the invention may be the same as the process according to the second aspect of the invention.

The Michael addition product which generates as a major by-product in the esterification step accumulates as a heavy matter in a high concentration on the bottom of a distillation column for recovering an effective ingredient.

In the fifth aspect of the invention, the decomposition reaction of the Michael addition product can be conducted by any of the continuous process, batch process, semi-batch process, intermittent-discharge process, and the like. However, the continuous process is preferred. The type of the reactor also is not particularly limited, and a reactor of any type can be employed, such as a flow-through type tubular reactor, thin-film flowing-down type reactor, complete mixing type stirring vessel reactor, circulating complete-mixing vessel reactor, or the like. For recovering a useful ingredient contained in the products of the decomposition reaction, use can be made of either a method in which the useful ingredient is obtained by evaporation or distillation during the reaction or a method in which after the decomposition reaction, the useful ingredient is obtained by evaporation or distillation. However, the former method, which is a reaction distillation technique, is preferred for obtaining a high recovery.

In the case where the reaction distillation technique is employed, the reaction pressure considerably depends on the reaction temperature which will be described later. A pressure is employed at which most of the useful ingredients which have been yielded by the decomposition reaction and of the useful ingredients which were contained in the feed material for the decomposition reaction, such as acrylic acid, an acrylic ester, and an alcohol, vaporize.

The catalyst is selected from inorganic acids such as sulfuric acid and phosphoric acid, organic acids such as methanesulfonic acid and p-toluenesulfonic acid, and the like. However, organic acids are preferred.

In the fifth aspect of the invention, the concentration of the acid catalyst is from 0.1 to 1.0% by weight, preferably from 0.2 to 0.8% by weight, based on the feed liquid.

The temperature for the decomposition reaction is preferably from 120 to 200° C. The liquid residence time as calculated from the amount of the liquid discharged is preferably from 0.5 to 50 hours, especially from 2 to 20 hours. In the case where the decomposition reaction is conducted continuously, the liquid residence time calculated from the amount of the liquid discharged can be regarded as the reaction time. For example, when the liquid capacity of the reactor is 500 L and the liquid discharge amount is 100 L/H, then the residence time is 5 hours.

Incidentally, the ordinary decomposition reaction conditions which have hitherto been employed include a p-toluenesulfonic acid concentration of from 5 to 15% by weight based on the feed liquid, a decomposition reaction temperature of from 180 to 230° C., and a reaction time of from 0.1 to 4.0 hours. The present inventors analyzed reactions yielding an ether as a by-product and decomposition reactions of Michael addition products from many angles, and have found that to use an acid as a catalyst in a low concentration and to employ a relatively low decomposition temperature are preferred for inhibiting the generation of ethers as by-products. Furthermore, the simultaneous treatment of the Michael addition product generated in the step of (meth)acrylic acid production is expected to produce an effect that the presence of acrylic acid and oligomers thereof diminishes the generation of an ether as a by-product, as described in JP-A-9-183752 and JP-A-9-183753. Moreover, the simultaneous en bloc treatment of the Michael addition product of (meth)acrylic acid has an advantage that the amount of the mixture treated per unit time period can be increased to heighten the rate of flow through the piping and, in particular, the discharge of residues having a high viscosity is facilitated.

When the decomposition reaction conditions according to the fifth aspect of the invention are employed, the progress of the decomposition reaction of Michael addition products becomes slightly slow. However, a sufficiently high recovery is obtained when the reaction time is prolonged to some degree.

The distillate obtained through the decomposition reaction, which is rich in (meth)acrylic acid, a (meth)acrylic ester, and an alcohol, is wholly recovered and sent to the step of acrylic ester production. Although the place to which the distillate recovered is sent is not particularly limited, it is preferred to send the recovered distillate to a step not after the step of separating light matters because the distillate contains light matters in a slight amount. One of the major advantages of the fifth aspect of the invention is that because heavy matters obtained as by-products in each of the step of (meth)acrylic acid production and the step of (meth)acrylic ester production can be treated en bloc and because the values recovered can be sent only to the step of (meth)acrylic ester production, the process can be simplified and a great contribution is made to a higher efficiency brought about by diminutions in construction cost, operational personnel, and utilities and to cost reduction.

### EXAMPLES

The invention will be explained below in more detail by reference to Examples.

### Example 1

As shown in FIG. 1, bottoms from a heavy-matter separation column equipped with a film evaporator as a reboiler in a methyl acrylate production step were used as a feed material to conduct a decomposition reaction. The bottoms had a composition consisting of 19% by weight acrylic acid, 1% by weight β-hydroxypropionic acid, 7% by weight methyl β-hydroxypropionate, 8% by weight β-acryloxypropionic acid, 6% by weight methyl β-acryloxypropionate, 40% by weight β-methoxypropionic acid, 11% by weight methyl β-methoxypropionate, and 8% by weight heavy matters and others. The bottoms were fed to a decomposition reactor at 865 kg/h. The decomposition reactor was a stirring vessel made of Hastelloy C having an inner diameter of 1,000 mm and a height of 2,000 mm, and a heat medium was supplied to an external jacket to regulate the reaction temperature to 200° C. The liquid residence time was regulated by controlling the liquid level in the decomposition reactor. The reaction pressure was kept at 500 kPa, which was a pressure necessary for maintaining a liquid phase. The rate of recycling to the reboiler for the heavy-matter separation column and the rate of residue discharge were regulated to 800 kg/h and 65 kg/h, respectively, so that the residence time calculated from liquid discharge amount became 10 hours. The operation could be stably continued over 3 months without arousing pipe clogging or other troubles. The composition of the residue was analyzed by gas chromatography and, as a result, found to consist of 0.6% by weight water, 10% by weight methanol, 11% by weight methyl acrylate, 44% by weight acrylic acid, 0.4% by weight β-hydroxypropionic acid, 4% by weight methyl β-hydroxypropionate, 2% by weight β-acryloxypropionic acid, 1% by weight methyl β-acryloxypropionate, 14% by weight β-methoxypropionic acid, 4% by weight methyl β-methoxypropionate, and 9% by weight heavy matters and others.

### Example 2

Bottoms from a heavy-matter separation column equipped with a film evaporator as a reboiler in a methyl acrylate production step were used as a feed material to conduct a decomposition reaction. The bottoms had a composition consisting of 20% by weight acrylic acid, 1% by weight β-hydroxypropionic acid, 8% by weight methyl β-hydroxypropionate, 8% by weight β-acryloxypropionic acid, 7% by weight methyl β-acryloxypropionate, 41% by weight β-methoxypropionic acid, 12% by weight methyl β-methoxypropionate, and 3% by weight heavy matters and others. The bottoms were fed to a decomposition reactor at 150 kg/h. The decomposition reactor was a stirring vessel made of Hastelloy C having an inner diameter of 1,000 mm and a height of 2,000 mm, and a heat medium was supplied to an external jacket to regulate the reaction temperature to 200° C. The reaction pressure was kept at 130 kPa. A column having an inner diameter of 400 mm and a height of 4,000 mm and packed with a packing to 2,000 mm and a condenser were connected to an upper part of the stirring vessel reactor to conduct the decomposition reaction by the reaction distillation technique. The liquid residence time was regulated by controlling the liquid level in the decomposition reactor so that the residence time calculated from liquid discharge amount became 10 hours. As a result, a clogging trouble occurred in a downstream part of the discharge piping after a 1-month continuous operation, and the operation was hence stopped. The rate of residue discharge during this period was 76 kg/h on-the average. The composition of the residue was analyzed by gas chromatography and, as a result, found to consist of 0.2% by weight water, 0.2% by weight methanol, 0.3% by weight methyl acrylate, 39% by weight acrylic acid, 0.3% by weight β-hydroxypropionic acid, 7% by weight methyl β-hydroxypropionate, 4% by weight β-acryloxypropionic acid, 4% by weight methyl β-acryloxypropionate, 31% by weight β-methoxypropionic acid, 8% by weight methyl β-methoxypropionate, and 6% by weight heavy matters and others.

The results of Example 1 and Example 2 clearly show the following. When the method of decomposition reaction according to the second aspect of the invention is applied to Michael addition products taken out of an acrylic ester purification step, not only the recovery of effective ingredients can be heightened as compared with the reaction distillation method heretofore in use, but also the residue contains a larger proportion of light matters. The residue hence has enhanced flowability, so that the clogging trouble can be avoided and continuous operation can be stably attained.

### Example 3

Bottoms from a rectifier in an n-butyl acrylate production step were subjected to a decomposition reaction.

The bottoms from the rectifier for n-butyl acrylate had a composition consisting of 16% by weight n-butyl acrylate, 59% by weight n-butyl β-n-butoxypropionate, 4% by weight n-butyl β-acryloxypropionate, 2% by weight n-butyl β-hydroxypropionate, and 19% by weight heavy matters and others. The bottoms were fed to a decomposition reactor at 580 g/h.

The decomposition reactor had an inner diameter of 200 mm and a length of 400 mm and was made of Hastelloy C. A distillation column having an inner diameter of 30 mm and a length of 1,000 mm and packed with a coil packing to 500 mm was installed above the reactor together with the attached condenser and vacuum system. The reaction temperature in the decomposition reactor was regulated with an external heater. The liquid residence time was regulated by controlling the liquid level in the decomposition reactor.

p-Toluenesulfonic acid was supplied as a decomposition reaction catalyst at 2.9 g/h (0.5% by weight based on the feed liquid), and the decomposition reaction was conducted at a reaction pressure of 47 kPa, decomposition temperature of 160° C., and residence time of 10 hours.

The composition of the residue discharged through the column bottom was analyzed by gas chromatography and, as a result, found to consist of 6% by weight n-butyl acrylate, 36% by weight n-butyl β-n-butoxypropionate, 2% by weight n-butyl acryloxypropionate, 0.3% by weight n-butyl β-hydroxypropionate, 1.4% by weight p-toluenesulfonic acid, and 54% by weight heavy matters and others. This reaction residue was obtained at 199.8 g/h. This reaction residue was ascertained to have high flowability.

A distillate comprising acrylic acid, n-butyl acrylate, and n-butanol as main components was recovered through the column top at 382.5 g/h. It contained di-n-butyl ether in an amount of 0.35% by weight.

### Example 4

Completely the same feed material and apparatus as in Example 3 were used, except that p-toluenesulfonic acid was supplied as a catalyst at 290 g/h (5% by weight based on the feed liquid). The feed material was fed at 5.80 kg/h. The decomposition reaction was conducted under the conditions of a reaction temperature of 200° C., pressure of 120 kPa, and residence time of 1 hour.

As a result, a reaction residue was obtained through the column bottom at 2.41 kg/h on the average. This reaction residue had slightly poorer flowability than that in Example 3. The reaction residue had a composition consisting of 4% by weight n-butyl acrylate, 34% by weight n-butyl β-n-butoxypropionate, 2% by weight n-butyl acryloxypropionate, 0.3% by weight n-butyl β-hydroxypropionate, 12% by weight p-toluenesulfonic acid, and 48% by weight others.

A distillate comprising acrylic acid, n-butyl acrylate, and n-butanol as main components was recovered through the top of the distillation column above the decomposition reactor at 3.68 kg/h on the average. It contained di-n-butyl ether in an amount of 2.78% by weight.

### Example 5

Using the same decomposition reactor as in Example 3, bottoms from a rectifier for heavy-matter separation in a methyl acrylate production plant were subjected to a decomposition reaction at a pressure of 60 kPa using the same catalyst kind, concentration, temperature, and liquid residence time as in Example 3. The feed material had a composition consisting of 20% by weight acrylic acid, 8% by weight β-acryloxypropionic acid, 12% by weight methyl β-methoxypropionate, 7% by weight methyl β-hydroxypropionate, 40% by weight β-methoxypropionic acid, 7% by weight methyl β-acryloxypropionate, and 6% by weight others. It was fed at 580 g/h.

As a result, a liquid recovered was obtained through the top of the distillation column above the decomposition reactor at 397 g/h on the average. Dimethyl ether was caught with an acetone-dry ice trap at 0.72 g/h.

### Example 6

A decomposition reaction was conducted using completely the same feed material and decomposition reactor as in Example 5, except that the catalyst concentration, reaction temperature, liquid residence time, and reaction pressure were changed to 5% by weight based on the feed material, 200° C., 1 hour, and 180 kPa, respectively. A liquid recovered was obtained through the top of the distillation column above the decomposition reactor at 3.87 kg/h on the average. Dimethyl ether was caught with the acetone-dry ice trap at 68.1 g/h.

A comparison between Example 3and Example 4 and a comparison between Example 5 and Example 6 clearly show that the generation of an ether compound is inhibited by regulating the feed amount of an acid catalyst so as to be in a specific range.

### Example 7

Bottoms from a rectifier (heavy-matter separation column), which had the composition shown below containing Michael addition products of methyl acrylate in a high concentration, and bottoms from an acrylic acid rectifier in an acrylic acid production step, which had the composition shown below, were used as feed materials to conduct a pyrolysis reaction. Incidentally, the methyl acrylate rectifier was one equipped with a film evaporator having a heating surface area of 2,000 cm² as a reboiler.

<Composition of Bottoms from Methyl Acrylate Rectifier>

Acrylic acid: 20% by weight

Methyl β-hydroxypropionate: 7% by weight

β-Acryloxypropionic acid: 8% by weight

Methyl β-acryloxypropionate: 7% by weight

β-Methoxypropionic acid: 40% by weight

Methyl β-methoxypropionate: 12% by weight

Heavy matters and others: 6% by weight

<Composition of Bottoms from Acrylic Acid Rectifier>

Acrylic acid: 21% by weight

β-Acryloxypropionic acid: 51% by weight

Heavy matters and others: 28% by weight

As a pyrolysis reactor was used a stirring vessel made of Hastelloy C having an inner diameter of 200 mm and a height of 400 mm. A heat medium was supplied to an external jacket to regulate the reaction temperature to 200° C. The liquid residence time was regulated by controlling the liquid level in the pyrolysis reactor. The reaction pressure was kept at 500 kPa, which was a pressure necessary for maintaining a liquid phase.

The bottoms from the methyl acrylate rectifier and the bottoms from the acrylic acid rectifier were fed to the pyrolysis reactor each at a rate of 500 g/hr. The products of the reaction were stored in an initial stage of the operation and then partly supplied to the film evaporator of the methyl acrylate rectifier in such a proportion that the recycling amount was 13 parts by weight per part by weight of the amount of the reaction products discharged from the pyrolysis reactor and sent outside the system. This film evaporator was operated at a pressure of 9.3 kPa and a temperature of 120° C., and the distillation residue was added to the two feed materials (the two kinds of bottoms from the respective rectifiers) and supplied to the pyrolysis reactor.

The whole system was thus stabilized, and the residence time in the pyrolysis reactor as calculated from liquid discharge amount was regulated to 10 hours.

As a result, the rate of recycling to the film evaporator of the methyl acrylate rectifier was 3.9 kg/hr and the rate of residue discharge was 300 g/hr (recycling proportion=92.9%). The distillate from the film evaporator was stably obtained at about 700 g/hr. The operation could be stably continued for 3 months without arousing troubles such as pipe clogging.

The composition of the residue discharged from the system was analyzed by gas chromatography, and the results thereof are as follows.

<Residue Composition>

Water: 0.5% by weight

Methanol: 6% by weight

Methyl acrylate: 7% by weight

Acrylic acid: 56% by weight

Methyl β-hydroxypropionate: 1% by weight

β-Acryloxypropionic acid: 6% by weight

Methyl β-acryloxypropionate: 1% by weight

β-Methoxypropionic acid: 5% by weight

Methyl β-methoxypropionate: 2% by weight

Heavy matters and others: 16% by weight

Namely, the recovery of values (amount recovered/all heavy matters supplied) was 70% by weight.

Furthermore, after the continuous operation for 3 months, the dimethyl ether which had caught with a dry ice-acetone trap disposed in the vacuum line of the film evaporator was analyzed and weighed. As a result, the amount thereof was 1.8 g.

### Example 8

The same two kinds of bottoms as those used as feed materials in Example 7 were fed to a rector each at 75 kg/hr. As the reactor was used a stirring vessel made of Hastelloy C having an inner diameter of 1,000 mm and a height of 2,000 mm. A heat medium was supplied to an external jacket to regulate the reaction temperature to 200° C. The reaction pressure was kept at 130 kPa. Furthermore, a column having an inner diameter of 400 mm and a height of 4,000 mm and packed with a packing to 2,000 mm and a condenser were connected to an upper part of the stirring vessel to conduct a decomposition reaction by the reaction distillation technique. The liquid residence time was regulated by controlling the liquid level in the decomposition reactor so that the residence time calculated from liquid discharge amount became 10 hours.

As a result, a downstream part of the discharge piping was slightly clogged in a one-month continuous operation, but a by-pass piping was used to cope with it. The rate of residue discharge during this period was 55 kg/hr on the average. The composition of the residue was analyzed by gas chromatography, and the results thereof are as follows.

<Residue Composition>

Water: 0.2% by weight

Methanol: 0.1% by weight

Methyl acrylate: 0.2% by weight

Acrylic acid: 15% by weight

Methyl β-hydroxypropionate: 3% by weight

β-Acryloxypropionate: 18% by weight

Methyl β-acryloxypropionate: 3% by weight

β-Methoxypropionic acid: 14% by weight

Methyl β-methoxypropionate: 4% by weight

Heavy matters and others: 43% by weight

Namely, the recovery of values (amount recovered/all heavy matters supplied) was 63% by weight.

The results of Example 7 and Example 8 clearly show the following. According to the method of the invention, not only the recovery of effective ingredients can be heightened as compared with the reaction distillation method heretofore in use, but also the residue contains a larger proportion of light matters. The residue hence has enhanced flowability, so that the clogging trouble can be avoided and continuous operation can be stably attained.

### Example 9

The same reactor as in Example 7 was used. To an upper part of the reactor were connected a distillation column having an inner diameter of 30 mm and a length of 1,000 mm and packed with a coil packing to 500 mm and the attached condenser, vacuum system, and acetone-dry ice trap. The same two kinds of bottoms as those used as feed materials in Example 7 were fed to the reactor each at 290 g/hr. p-Toluenesulfonic acid was used as a decomposition catalyst in an amount of 5% by weight based on the feed materials. A decomposition reaction was conducted for 24 hours at a reaction temperature of 160° C. and a reaction pressure of 60 kPa for a residence time calculated from liquid discharge amount of 10 hours.

A liquid recovered was obtained through the top of the distillation column above the decomposition reactor at 396 g/hr on the average. Dimethyl ether was caught with the acetone-dry ice trap at a rate of 3.8 g per hour on the average.

The results of Example 7 and Example 9 clearly show the following. By conducting the pyrolysis reaction substantially in a liquid phase and further regulating the amount of an acid catalyst so as to be in a specific range, not only the recovery of effective ingredients can be heightened but also the generation of a methanol-derived ether is significantly inhibited.

### Example 10

Bottoms from a rectifier in an n-butyl acrylate production step and bottoms from a rectifier for heavy-matter separation in an acrylic acid production step were subjected to a decomposition reaction.

The bottoms from the rectifier for n-butyl acrylate had a composition consisting of 16% by weight n-butyl acrylate, 59% by weight n-butyl β-n-butoxypropionate, 4% by weight n-butyl β-acryloxypropionate, 2% by weight n-butyl β-hydroxypropionate, and 19% by weight other heavy matters. This feed material was fed to a decomposition reactor at 290 g/h.

The bottoms from the rectifier for heavy-matter separation from acrylic acid had a composition consisting of 21% by weight acrylic acid, 51% by weight β-acryloxypropionic acid, and 28% by weight other heavy matters. This feed material was simultaneously fed to the decomposition reactor at 290 g/h.

The decomposition reactor had an inner diameter of 200 mm and a length of 400 mm and was made of Hastelloy C. A distillation column having an inner diameter of 30 mm and a length of 1,000 mm and packed with a coil packing to 500 mm was installed above the reactor together with the attached condenser and vacuum system. The reaction temperature in the decomposition reactor was regulated with an external heater. The liquid residence time was regulated by controlling the liquid level in the decomposition reactor.

p-Toluenesulfonic acid was supplied as a decomposition reaction catalyst at 2.9 g/h (0.5% by weight based on the feed liquids), and the decomposition reaction was conducted at a reaction pressure of 47 kPa and a decomposition temperature of 160° C. for a residence time of 10 hours.

The composition of the residue discharged through the column bottom was analyzed by gas chromatography and, as a result, found to consist of 8.4% by weight acrylic acid, 1.0% by weight n-butyl alcohol, 5.1% by weight n-butyl acrylate, 18.3% by weight n-butyl β-n-butoxypropionate, 1.3% by weight n-butyl β-acryloxypropionate, 0.7% by weight n-butyl β-hydroxypropionate, 11.7% by weight β-acryloxypropionic acid, 1.4% by weight p-toluenesulfonic acid, and 52.1% by weight other heavy matters. This reaction residue was obtained at 199 g/h.

A distillate comprising 0.13% by weight water, 46.2% by weight acrylic acid, 33.2% by weight n-butyl acrylate, 13.0% by weight n-butanol, and 7.3% by weight others was recovered through the column top at 383 g/h. It contained di-n-butyl ether in an amount of 0.15% by weight.

### Example 11

Using completely the same apparatus as in Example 10, bottoms from a rectifier for n-butyl acrylate were subjected as the only feed material to a decomposition reaction experiment. This feed material was the same as in Example 10, and fed at 580 g/h to conduct a decomposition reaction. The other conditions were completely the same as in Example 10. The composition of the residue discharged through the column bottom was analyzed by gas chromatography and, as a result, found to consist of 6% by weight n-butyl acrylate, 36% by weight n-butyl β-n-butoxypropionate, 2% by weight n-butyl acryloxypropionate, 0.3% by weight n-butyl β-hydroxypropionate, 1.4% by weight p-toluenesulfonic acid, and 54.3% by weight others. This reaction residue was obtained at 199.8 g/h.

A distillate comprising acrylic acid, n-butyl acrylate, and n-butanol as main components was recovered through the column top at 382.5 g/h. It contained di-n-butyl ether in an amount of 0.35% by weight.

It was ascertained from Example 10 and Example 11 that even when heavy matters which have been obtained in an acrylic acid production step and contain Michael addition products in a high concentration are treated simultaneously, values can be satisfactorily recovered likewise and the generation of di-n-butyl ether as a by-product can be diminished.

### Example 12

Completely the same feed materials as in Example 10 were used, except that p-toluenesulfonic acid was supplied as a catalyst at 290 g/h (5% by weight based on the feed liquids). The feed materials were fed at 5.80 kg/h. A decomposition reaction was conducted under the conditions of a reaction temperature of 200° C., pressure of 120 kPa, and residence time of 1 hour.

As a result, a reaction residue was obtained through the column bottom at 2.3 kg/h on the average. A distillate comprising acrylic acid, n-butyl acrylate, and n-butanol as main components was recovered through the top of the distillation column above the decomposition reactor at 3.8 kg/h on the average. It contained di-n-butyl ether in an amount of 1.54% by weight.

### Example 13

The same decomposition reactor as in Example 10 was used. A feed material prepared by mixing bottoms from a rectifier for heavy-matter separation in a methyl acrylate production plant with heavy matters (bottoms from a rectifier) from an acrylic acid production plant in a ratio of 1:1 was used to conduct a decomposition reaction at a pressure of 60 kPa using the same catalyst kind, concentration, temperature, and liquid residence time as in Example 10. The feed material had a composition consisting of 21% by weight acrylic acid, 30% by weight β-acryloxypropionic acid, 6% by weight methyl β-methoxypropionate, 4% by weight methyl β-hydroxypropionate, 21% by weight β-methoxypropionic acid, 4% by weight methyl β-acryloxypropionate, and 14% by weight other heavy matters. It was fed at 580 g/h.

As a result, a liquid recovered was obtained through the top of the distillation column above the decomposition reactor at 396 g/h on the average. Dimethyl ether was caught with an acetone-dry ice trap at 0.35 g/h.

### Example 14

A decomposition reaction was conducted using completely the same feed material, decomposition reactor, and reaction conditions as in Example 13, except that the catalyst concentration was changed to 5% by weight based on the feed material amount. A liquid recovered was obtained through the top of the distillation column above the decomposition reactor at 397 g/h on the average. Dimethyl ether was caught with the acetone-dry ice trap at 3.8 g/h.

While the invention has been described in detail and with reference to specific embodiments thereof, it will be apparent to one skilled in the art that various changes and modifications can be made therein without departing from the spirit and scope thereof.

This application is based on a Japanese patent application filed on Nov. 28, 2001 (Application No. 2001-362895), Japanese patent application filed on Nov. 28, 2001 (Application No. 2001-362896), Japanese patent application filed on Nov. 28, 2001 (Application No. 2001-362897), and Japanese patent application filed on Dec. 25, 2001 (Application No. 2001-392057), the entire contents thereof being hereby incorporated by reference.

<Industrial Applicability>

As described above, according to the first and second aspects of the invention, Michael addition products generated as by-products in a (meth)acrylic acid compound or (meth)acrylic ester production step are pyrolyzed, whereby a (meth)acrylic ester can be recovered at a high recovery. Furthermore, the (meth)acrylic ester can be stably produced without causing a clogging trouble in the process. According to the third aspect of the invention, Michael addition products generated as by-products in a (meth)acrylic ester production step are subjected to a decomposition treatment using an acid as a catalyst, whereby (meth)acrylic acid, a (meth)acrylic ester, and an alcohol can be recovered at a high recovery. Furthermore, the generation of an ether as a by-product, which is problematic to the process and/or product quality, can be inhibited. According to the fourth aspect of the invention, which is a method of decomposing by-products of (meth)acrylic acid compound production, the step of decomposing by-products of (meth)acrylic acid production and the-step of decomposing by-products of (meth)acrylic ester production can be united into one to thereby bring about considerable economical effects such as power saving and a reduction in construction cost and energy. In addition, (meth)acrylic acid, a (meth)acrylic ester, and an alcohol can be stably recovered at a high recovery while inhibiting the generation of an alcohol-derived ether or olefin as a by-product. According to the fifth aspect of the invention, Michael addition reaction products generated as by-products in a (meth)acrylic acid production step and a (meth)acrylic ester production step are subjected en bloc to a decomposition treatment using an acid as a catalyst, whereby (meth)acrylic acid, a (meth)acrylic ester, and an alcohol can be recovered at a high recovery. Furthermore, the generation of an ether as a by-product, which is problematic to the process and/or product quality, can be inhibited. According to the fifth aspect of the invention, the steps of the decomposition reaction of Michael addition products can be united into one to thereby bring about considerable economical effects such as power saving and a reduction in construction cost and utility cost.

1. A process for producing a (meth)acrylic acid compound which has both (meth)acrylic acid production facilities and (meth)acrylic ester production facilities and in which by-products taken out of a purification step for purifying a reaction mixture of a (meth)acrylic ester are pyrolyzed to recover the (meth)acrylic ester therefrom, characterized in that the pyrolysis reaction of the by-products is conducted substantially in a liquid phase and at least part of the products of the pyrolysis reaction are returned to a (meth)acrylic ester purification step.

2. The process as claimed in claim 1, characterized in that at least 50% of the pyrolysis reaction products are returned to the purification step.

3. The process as claimed in claim 1, characterized in that the by-products are bottoms from a distillation column for separating heavy matters in a (meth)acrylic ester purification step.

4. The process as claimed in claim 1, characterized in that the pyrolysis reaction of the by-products is conducted in the presence of an acid catalyst and the acid catalyst is added in an amount of from 0.1 to 1.0% by weight based on the by-products.

5. The process as claimed in claim 1, characterized in that the by-products to be subjected to pyrolysis reaction are a mixture of by-products of (meth)acrylic acid production and by-products of (meth)acrylic ester production.

6. The process as claimed in claim 5, characterized in that the by-products of (meth)acrylic acid production are bottoms from a rectifier for separating heavy matters in a (meth)acrylic acid purification step and the by-products of (meth)acrylic ester production are bottoms from a rectifier for separating heavy matters in a (meth)acrylic ester purification step.

7. The process as claimed in claim 5, characterized in that the mixture of by-products of (meth)acrylic acid production and by-products of (meth)acrylic ester production is pyrolyzed in the presence of an acid catalyst and the acid catalyst is added in an amount of from 0.1 to 1.0% by weight based on the mixture.

8. The process as claimed in claim 1, characterized in that the rectifier for separating heavy matters in a (meth)acrylic ester purification step is equipped with a film evaporator as a reboiler.

9. The process as claimed in claim 1, characterized in that at least 80% of the pyrolysis reaction products are returned to a (meth)acrylic ester purification step.

10. The process as claimed in claim 1, characterized in that the temperature for the pyrolysis reaction is from 120 to 280° C. and the time period of the pyrolysis reaction is from 0.5 to 50 hours.

11. A process for producing a (meth)acrylic ester which comprises a (meth)acrylic ester-yielding reaction step and a step in which by-products separated from the step of yielding are pyrolyzed to recover a (meth)acrylic ester therefrom, characterized in that the pyrolysis reaction is conducted substantially in a liquid phase and at least 50% of the products of the pyrolysis reaction are returned to an upstream step.

12. The process for producing a (meth)acrylic ester as claimed in claim 11, characterized in that the by-products of the (meth)acrylic ester-yielding reaction are bottoms from a distillation column for separating heavy matters in a purification step for purifying the (meth)acrylic ester yielded.

13. The process for producing a (meth)acrylic ester as claimed in claim 12, characterized in that the distillation column is equipped with a film evaporator as a reboiler.

14. The process for producing a (meth)acrylic ester as claimed in claim 11, characterized in that the by-products of the (meth)acrylic ester-yielding reaction comprise a Michael addition product formed by the addition of water, methanol, ethanol, butanol, or (meth)acrylic acid to the α-position or β-position of a (meth)acryloyl group.

15. The process for producing a (meth)acrylic ester as claimed in claim 11, characterized in that the temperature for the pyrolysis reaction is from 120 to 280° C. and the time period of the pyrolysis reaction is from 0.5 to 50 hours.

16. The process for producing a (meth)acrylic ester as claimed in claim 11, characterized in that at least 80% of the pyrolysis reaction products are returned to an upstream step.

17. A method of decomposing by-products of (meth)acrylic ester production which comprises decomposing the by-products of (meth)acrylic ester production in the presence of an acid catalyst, characterized in that the acid catalyst is added in an amount of from 0.1 to 1.0% by weight based on the by-products.

18. The method of decomposing by-products of (meth)acrylic ester production as claimed in claim 17, characterized in that the by-products of (meth)acrylic ester production are bottoms from a rectifier for separating heavy matters in a (meth)acrylic ester purification step.

19. The method of decomposing by-products of (meth)acrylic ester production as claimed in claim 17, characterized in that the by-products of (meth)acrylic ester production comprise a Michael addition product formed by the addition of water, methanol, ethanol, n-butanol, or (meth)acrylic acid to the α-position or β-position of a (meth)acryloyl group.

20. The method of decomposing by-products of (meth)acrylic ester production as claimed in claim 17, characterized in that the temperature for the decomposition treatment is from 120 to 200° C. and the time period of the decomposition treatment is from 0.5 to 20 hours.

21. A method of decomposing by-products of (meth)acrylic acid compound production which comprises pyrolyzing a mixture of by-products of (meth)acrylic acid production and by-products of (meth)acrylic ester production in a liquid phase, characterized in that at least 50% of the products of the pyrolysis reaction are returned to a (meth)acrylic ester production step.

22. The method of decomposing by-products of (meth)acrylic acid compound production as claimed in claim 21, characterized in that the by-products of (meth)acrylic acid production are bottoms from a rectifier for separating heavy matters in a (meth)acrylic acid purification step and the by-products of (meth)acrylic ester production are bottoms from a rectifier for separating heavy matters in a (meth)acrylic ester purification step.

23. The method of decomposing by-products of (meth)acrylic acid compound production as claimed in claim 22, characterized in that the rectifier for separating heavy matters in a (meth)acrylic ester purification step is equipped with a film evaporator as a reboiler.

24. The method of decomposing by-products of (meth)acrylic acid compound production as claimed in claim 21, characterized in that the mixture of by-products of (meth)acrylic acid production and by-products of (meth)acrylic ester production comprises a Michael addition product formed by the addition of water, an alcohol, or (meth)acrylic acid to the α-position or β-position of a (meth)acryloyl group.

25. The method of decomposing by-products of (meth)acrylic acid compound production as claimed in claim 21, characterized in that the temperature for the pyrolysis reaction is from 120 to 280° C. and the time period of the pyrolysis reaction is from 0.5 to 50 hours.

26. The method of decomposing by-products of (meth)acrylic acid compound production as claimed in claim 21, characterized in that at least 80% of the pyrolysis reaction products are returned to a (meth)acrylic ester production step.

27. A method of decomposing by-products of (meth)acrylic acid compound production which comprises decomposing a mixture of by-products of (meth)acrylic acid production and by-products of (meth)acrylic ester production in the presence of an acid catalyst, characterized in that the acid catalyst is added in an amount of from 0.1 to 1.0% by weight based on the mixture.

28. The method of decomposing by-products of (meth)acrylic acid compound production as claimed in claim 27, characterized in that the by-products of (meth)acrylic acid production are bottoms from a rectifier for separating heavy matters in a (meth)acrylic acid purification step and the by-products of (meth)acrylic ester production are bottoms from a rectifier for separating heavy matter in a (meth)acrylic ester purification step.

29. The method of decomposing by-products of (meth)acrylic acid compound production as claimed in claim 27, characterized in that the mixture of the by-products of (meth)acrylic acid production and the by-products of (meth)acrylic ester production comprises a Michael addition product formed by the addition of water, an alcohol, or (meth)acrylic acid to the α-position or β-position of a (meth)acryloyl group.

30. The method of decomposing by-products of (meth)acrylic acid compound production as claimed in claim 27, characterized in that the temperature for the decomposition treatment is from 120 to 200° C. and the time period of the decomposition treatment is from 0.5 to 20 hours.
